(19) United States
(12) Patent Application Publication
(10) Pub. No.: US 2008/0254074 A1
(43) Pub. Date: Oct. 16, 2008

(54) COMPOSITION AND METHOD FOR REGULATING SEBUM FLOW

(75) Inventors: Anne Dussaud, Tarrytown, NY (US); Pradeep Yadav, Waterbury, CT (US)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., D/B/A UNILEVER, Englewood Cliffs, NJ (US)

(21) Appl. No.: 11/734,078

(22) Filed: Apr. 11, 2007

(51) Int. Cl. A61K 8/81; A61K 8/87; A61K 8/88

(52) U.S. Cl. 424/401; 424/489; 424/724; 424/78.08; 514/789

(57) ABSTRACT

A skin care composition for regulating sebum flow from sebocytes is described. The composition comprises spheroids, and optionally, thickening agents and when a spheroid and sebum mixture is made upon application, the resulting mixture leaves a pleasant, silky and powdery sensation.

### FIELD OF THE INVENTION

The present invention is directed to a skin care composition and a method for regulating sebum flow. More particularly, the invention is directed to a skin care composition comprising spheroids, and optionally, a thickening agent. The skin care composition of this invention displays excellent oil absorbing characteristics, and surprisingly, does not create a sticky and greasy feeling when applied to skin. The skin care composition of this invention is easy to apply and yields a pleasant, silky and powdery sensation that is long lasting after being applied. Furthermore, the skin care composition of this invention displays excellent sebum wicking properties when applied to skin.

### BACKGROUND OF THE INVENTION

Cosmetic products or skin compositions, which improve the appearance of skin, are increasingly popular with consumers. For example, many consumers seek to minimize the “oily skin” look, resulting from sebum production in the sebaceous glands of the skin. Moreover, consumers also seek to alleviate or delay the signs of aged or photo-aged skin as well as dry and sagging skin. Further, consumers are concerned with the degree of pigmentation of their skin, whereby people with age spots or freckles often wish to eliminate or reduce the pronouncement of such spots. Others may wish to lighten their natural skin or reduce skin darkening caused by exposure to sunlight. To meet the needs of consumers, many attempts have been made to develop products that improve skin characteristics. The products developed thus far, however, tend to have low efficacy or undesirable side effects, such as, for example, toxicity or skin irritation. Additionally, known products are not always useful at improving a variety of negative skin characteristics, like those associated with dryness, color, and especially, excess oil.

The texture of skin, like skin on the face, is genetically encoded and consists of an ordered network of grooves. Such a network is present at birth and evolves as one gets older. Typically, the grooves, which make up the network, are V-shaped and a few tenths of a micron in depth. Moreover, it is believed that a surface created by the network of grooves creates channels for transporting fluids like sebum supplied from sebaceous ducts and sweat secreted by sweat glands. When oils are applied to skin, voids (i.e., the volume created by the grooves) allow for the reduction of a shiny look since oil on the skin fills the voids and is less visible than oil on peak or plateau regions of the skin. In the T-zone of the face, the portion of the face where the concentration of sebaceous glands is highest, the skin has lost most of its network of grooves. Such a loss often results in a reduction of sebum transport and the enhancement of a shiny appearance. It can also result in a reduction in pore drainage, unhealthy pores and infection.

There is an increasing interest to develop a skin care composition that, at the very least, decreases the impact of sebum secretion from sebocytes, especially in the facial area. Compositions which help restore the transport properties of skin are very desirable not only to reduce shininess but also to assist in the formation of uniform films of sebum in order to ensure skin moisturization and minimize any ailment associated with oily skin. This invention, therefore, is directed to a skin care composition and a method for regulating sebum flow. The skin care composition of the present invention, surprisingly and at the very least, decreases the impact of sebum secretion from sebocytes and can be formulated to, for example, reduce wrinkles, moisturize and/or lighten skin. Moreover, the skin care composition of the present invention is not sticky or tacky, and does not create a draggy sensation when applied. The same, when applied, yields a pleasant, silky and powdery sensation (that is long lasting) as well as excellent sebum wicking characteristics.

### Additional Information

Efforts have been disclosed for making anti-sebum skin care cosmetic compositions. In European Patent EP 1181007 B1, anti-sebum skin care cosmetic compositions with branched esters are described.

Other efforts have been disclosed for alleviating oily skin. In U.S. Application Publication No. 2005/0079144 A1, methods for decreasing sebum production are described.

Still other efforts have been disclosed for treating skin. In International Application No. WO 94/27569, a skin treatment method that utilizes a composition and a pad is described.

U.S. Application No. 2006/0189495 A1 discloses compositions with suspended particles that display high stability.

None of the additional information above describes a skin care composition that, at the very least and surprisingly, decreases the impact of sebum secretion from sebocytes, and unexpectedly, yields a pleasant, silky and powdery sensation after being applied, even when a thickening agent is employed. Moreover, none of the additional information describes a skin care composition having excellent wicking properties as defined herein.

### SUMMARY OF THE INVENTION

In a first aspect, the present invention is directed to a skin care composition comprising:
(a) 1 to 40% by weight spheroid, the spheroid comprising an approximate diameter from about 3.5 to about 60 microns;
(b) non-volatile fluid;
(c) cosmetically acceptable carrier; and
(d) optionally, a thickening agent,
   the spheroid and optionally the thickener when mixed with sebum results in a sebum mixture wherein the spheroid, sebum and optional thickening agent in the mixture each, independently, have a volume fraction at a ratio, ε, that is greater than or equal to about 2.5 and less than about 10, where

ε=(C_{B}·V_{B})/(C_{A}/P_{A}+C_{C}/P_{C}),

C_{A} is weight percent of the thickening agent, C_{B} is weight percent of the spheroid, C_{C} is weight percent of sebum plus non-volatile fluid, V_{B} is the inverse of the bulk density of the spheroid (cm³/g), P_{A} is density of the thickening agent, and P_{C} is density of sebum,
and wherein the mixture has a shear elastic modulus, Gₒ, ranging from about 1×10³ to less than about 9×10⁷ Pascals where the non-volatile fluid is present at a weight percent in grams that is ±15% of an oil absorption number of the spheroid.

In a second aspect, the present invention is directed to a method for treating skin with the skin care composition of the first aspect of this invention.

Additional aspects of the present invention will more readily become apparent from the description and examples which follows.

Shear elastic modulus, Gₒ, means the description of a substance's tendency to be deformed when a force is applied to it in the linear visco-elastic region at about ambient temperature, the slope of a substance's stress strain curve. Shear elastic modulus can be measured with a rheometer (like an Ares Rheometer) supplied from Rheometrics and utilizing art recognized strain sweep tests on compositions at ambient temperature. Measurement of shear elastic modulus is further defined (and techniques for measuring the same are demonstrated) in Non-Newtonian Fluid Mechanics, 24 (1987) 183, Yates, J., and C.R. Physique 4 (2003) 221 M. Cloitre, the disclosures of which is incorporated herein by reference. Samples used for the rheological measurement can be prepared by mixing oil and spheroid, or oil, spheroid and thickener. Such samples (having different ratios of oil to spheroid/thickener) are suitable for Gₒ measurement. A preferred oil used to determine Gₒ is artificial sebum like the formula described in Example 1. Other preferred oils suitable for use include those with a viscosity ranging from about 3.5 cps to 100 cps at ambient temperature and measured with a Ubbelohde Viscometer.

Skin care composition, as used herein, is meant to include cosmetic compositions suitable for use with humans and suitable to enhance a skin characteristic. Such a composition may generally be classified as leave-on or rinse off and is meant to include a base or end use compositions which include but are not limited to hair care compositions like shampoos; conditioners or tonics; lipsticks; leave on skin enhancers, and color cosmetics. The preferred form of the skin care composition of this invention is, however, a leave on composition that in addition to regulating sebum flow also yields a pleasant, silky and powdery sensation after being applied. Spheroid, as used herein, is meant to include any particle that is preferably solid and that is preferably spherical in shape, ellipsoidal in shape or fiber-like (typically 50 to 650, and preferably, 100-500 and most preferably, 200 to 400 microns in length). Approximate diameter is meant to mean the largest diameter suitable to be measured on the spheroid. Non-volatile, as used herein, means a substance that does not sublime or evaporate at standard temperature and pressure (e.g., a vapor pressure in mm Hg of less than 0.005 at 25° C.). Excellent wicking properties means 1 microliter of sebum on a 37 micron film of skin care base composition (i.e., comprising water to balance, about 4% by weight stabilizer like Sepigel {polyacrylamide, a C₁₃-C₁₄ isoparrafin and laureth-7} made available from Seppic, about 5% by weight C₁₂H₂₆ as well as spheroid, optionally thickening agent, and non-volatile fluid such that the weight percent in grams of non-volatile fluid is ±15% of the spheroid absorption number) expands or dissipates to a final area (A^{f}) that is at least about 250%, and preferably, at least about 350% larger than an initial area, (Aⁱ), where (Aⁱ) is the area of sebum when initially placed on the film and (A^{f}) is the final area of sebum on the film taken after about 30 minutes and at about ambient temperature. In a most preferred embodiment, excellent wicking means A^{f} is at least about 1500 percent greater than Aⁱ after 1 microliter of sebum is on the film for about 2 hours and at about ambient temperature.

All wicking properties are determined by using skin care compositions consistent with this invention and the sebum and wicking experiments are further described in Liquid Transport in the Networked Microchannels of the Skin Surface by Dussaud et al. (Langmuir 2003, 19, 7341-7345), the disclosure of which incorporated herein by reference.

The skin care composition of the present invention can be in the form of a mousse, liquid, lotion, cream, gel, soap bar, toner, semi-solid, stick, ointment or face mask, but, again, is preferably a leave on lotion or cream. Skin, as used herein, is meant to include skin on the face, neck, chest, back, arms, hands, legs and scalp.

All ranges identified herein are meant to implicitly include all ranges subsumed therein if, for example, reference to the same is not explicitly made. All percents by weight, unless otherwise stated, are based on total weight of the skin care composition.

### DETAILED DESCRIPTION OF THE PREFERRED EMBODIMENTS

The only limitation with respect to the type of spheroid suitable for use in this invention is that the spheroid is one which may be used in a skin care composition and yield a shear elastic modulus in a sebum mixture as defined above. General examples of the types of spheroids suitable for use in this invention include silica modified ethylene/methacrylate copolymer microspheres, talc modified ethylene/methacrylate copolymer microspheres, mixtures thereof or the like. Other examples of the types of spheroids suitable for use in this invention include those comprising polyolefins like polyethylene, polypropylene and/or polybutylene-based polymers, polyamides (like nylon fibers), mixtures thereof or the like. Still other preferred spheroids suitable for use in this invention include those comprising polyurethane, polystyrene, epoxy resins, urea resins, silicone resins, mixtures thereof or the like.

In a preferred embodiment, the spheroids used in this invention comprise polyethylenes, or are talc comprising particles or mixtures thereof. The former are often sold under the names Cerapure (made commercially available by Shamrock), Asensa (made commercially available by Honeywell) and Miperon (made commercially available by Mitsui Petrochemical Industries, Ltd.). Another preferred polyethylene-based spheroid is sold under the name CL-2080 (made commercially available by Kobo Industries). Other preferred spheroids suitable for use in this invention include nylons (e.g., nylon-12) sold under the name SP-10 which is made commercially available by Kobo Industries Still other preferred spheroids suitable for use in this invention include those comprising copolymers of ethylene and methacrylate that contain silica or talc and sold under the names SPCAT-I2 and DSPCS-I2, respectively, both of which are also made commercially available by Kobo Industries. Other spheroids comprising polystyrenes and polymethyl methacrylate (sold, for example, under the names Ganzpearl GS-0605 and GME0380, respectively) and made available from Presperse are also often preferred.

Even other spheroids suitable for use in this invention include natural polymeric spheroids like those which comprise starch and those which comprise silk, the former, for example, made available from National Starch and Chemical and the latter, for example, made available by Engelhard Corporation. Still other natural polymeric spheroids suitable for use in this invention include those natural polymeric spheroids comprising cellulose such as Celluflow and Cellulo Beads, the former made commercially available by Chisso Corporation and the latter made available by Kobo Industries.

When selecting spheroids for use in this invention, typically they often desired have an oil absorption number from about 0.2 to about 15 g/g, and preferably, from about 0.2 to about 12 g/g, and most preferably, from about 0.9 to about 8 g/g, including all ranges subsumed therein, where g/g means gram of sebum absorbed per gram of spheroid at ambient temperature.

In a more preferred embodiment of this invention, the spheroids employed make up from about 1 to about 15, and most preferably, from about 5 to about 10% by weight of the skin care composition. In another more preferred embodiment, the spheroids have an approximate diameter from about 4 to about 40, and most preferably, from about 5 to about 35 microns, including all ranges subsumed therein. Optionally, but often preferably, the spheroids employed in this invention are used with thickening agents that are suitable to thicken oil (i.e. sebum) at the temperature of the skin that the skin care composition is applied to. Illustrative but non-limiting examples of the types of thickening agents suitable for use in this invention include bismuth oxychloride, mica, fumed silica, micronized teflon, aluminum silicate, bentonite, calcium silicate, chalk, clay (like kaolin and laponite), hydrated silica zinc oxide, silica, talc, mica, titanium dioxide, magnesium oxide, alumina, calcium carbonate, mixtures thereof or the like. In a preferred embodiment, the thickening agent employed has an approximate diameter from about 0.5 to about 3.5, more preferably, from about 0.7 to about 2.5, and most preferably, from about 0.8 to about 1 micron, including all ranges subsumed therein.

In a most preferred embodiment, the amount of thickening agent used is about equal to, to at least about 15 times less than, and preferably, about equal to, to less than about 5 times less than, and most preferably, from about equal to, to about 2 times less than the spheroid concentration in the mixture of spheroid, thickener and sebum. In an especially preferred embodiment, the thickening agent employed in this invention is fumed silica.

In an often preferred embodiment, Gₒ˜A^{p} where A is the weight of spheroid and any optional thickener in the sebum mixture and exponent p is preferably an integer of at least about 5, and most preferably, from about 10 to about 35. Moreover, and especially when a thickening agent is employed, Gₒ is preferably from about 1×10³ to about 5×10⁵, and most preferably, from about 4×10³ to about 1×10⁵ Pascals, including all ranges subsumed therein

It should be known that commercially acceptable and conventional vehicles may be used, acting as diluants, dispersants or carriers for the spheroids and thickeners used as well as for any optional additives used in the compositions of this invention. Therefore, the vehicle of the skin care composition described herein may be aqueous-based, anhydrous or an emulsion whereby a water-in-oil or oil-in-water emulsion is generally preferred. If the use of water is desired, water typically makes up the balance of the skin care composition, and preferably, makes up from about 5 to about 98%, and most preferably, from about 65 to about 90% by weight of the skin benefit composition, including all ranges subsumed therein.

In addition to water, organic solvents may be optionally included to act or assist as carriers within the compositions of the present invention. Illustrative and non-limiting examples of the types of organic solvents suitable for use in the present invention include alkanols like methyl, ethyl and isopropyl alcohol, mixtures thereof or the like.

Other optional additives suitable for use include ester oils like isopropyl myristate, cetyl myristate, 2-octyldodecyl myristate, avocado oil, almond oil, olive oil, neopentylglycol dicaprate, mixtures thereof or the like.

Emollients may also be used, if desired, in the skin care composition of the present invention. Alcohols like 1-hexadecanol (i.e., cetyl alcohol) and phenoxyethanol are often desired as are the emollients generally classified as silicone oils and synthetic esters. Silicone oils suitable for use include cyclic or linear polydimethylsiloxanes containing from 3 to 9, preferably from 4 to 5, silicon atoms. Linear volatile silicone materials generally have viscosities less than about 5 centistokes at 25° C. while cyclic materials typically have viscosities of less than about 10 centistokes. Nonvolatile silicone oils useful as an emollient material in the inventive skin benefit composition described herein include polyalkyl siloxanes, polyalkylaryl siloxanes and polyether siloxane copolymers. The essentially non-volatile polyalkyl siloxanes useful herein include, for example, polydimethylsiloxanes with viscosities of from about 5 to about 25 million centistokes at 25° C. Among the preferred non-volatile emollients useful in the present compositions are the polydimethylsiloxanes having viscosities from about 10 to about 400 centistokes at 25° C.

The ester emollients that may optionally be used are:
(1) alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.
(2) ether-esters such as fatty acid esters of ehtoxylated fatty alcohols.
(3) polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) wax esters such as beeswax, spermaceti, stearyl stearate and arachidyl behenate.
(5) sterols esters, of which cholesterol fatty acid esters are examples.

Emollients are optional, but volatile emollients can make up from about 0.1 to about 50% by weight of the skin care composition, including all ranges subsumed therein.

Humectants of the polyhydric alcohol type may also be employed in the skin care compositions of this invention. The humectant often aids in increasing the effectiveness of the emollient, reduces scaling, stimulates removal of built-up scale and improves skin feel. Typical polyhydric alcohols include glycerol, polyalkylene glycols and more preferably alkylene polyols and their derivatives, including propylene glycol, methyl propylene diol, dipropylene glycol, polypropylene glycol, polyethylene glycol and derivatives thereof, hydroxypropyl sorbitol, hexylene glycol, glycerin, sorbitol, 1,3-butylene glycol, 1,2,6-hexanetriol, ethoxylated glycerol, propoxylated glycerol and mixtures thereof. The amount of humectant may often range anywhere from 0.2 to 15%, and preferably, from about 0.5 to about 3.0% by weight of the skin care composition, including all ranges subsumed therein.

However, and again, non-volatile fluids used in the skin care composition of this invention should be present at a weight percent that is ±15% of the spheroid absorption number to enhance moisturization. Volatile fluids should be added to the skin care composition to enhance wicking. When a non-whitening composition is desired, the volatile fluid in the skin care composition should be enough such that it should last when the composition is applied but evaporate before skin sebum secretion begins so that the refractive indexes of skin and the skin care composition are similar enough to yield a non-whitening effect. In an especially preferred embodiment, volatile fluids (those with, for example, a vapor pressure from about 0.01 to about 0.09 mmHg at 25° C.) should often make up from about 5 to about 15 percent by weight of the skin care composition, including all ranges subsumed therein.

Fatty acids suitable for use in the skin care composition of this invention are art recognized and include, for example, 10-30 carbon fatty acids such as petroselinic, linoleic, stearic, hydroxystearic, oleic, ricinoleic, arachidic, behenic, erucic acid, mixtures thereof, or the like.

Cellulose-based additives and natural gums may also be utilized as part of the cosmetically acceptable carrier in the skin care compositions of the present invention. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Polyacrylic acid-based additives (like Carbopol®) and Sepigel are stabilizers which may be used. Amounts of these types of additives, if used, may range from 0.0001 to 7%, usually from 0.001 to 6%, optimally from 0.01 to 5% by weight, based on total weight of the skin care composition, including all ranges subsumed therein.

Collectively, the water, solvents, silicones, esters, humectants, fatty acids and/or cellulose-based and natural gum additives will constitute the cosmetically acceptable carrier in amounts from 1 to 99.9%, preferably from 80 to 99% by weight.

Various types of optional additional active ingredients may be used in the skin care compositions of the present invention. Actives are defined as skin benefit agents other than emollients and other than ingredients that merely improve the physical characteristics of the composition. Although not limited to this category, general examples include alpha-hydroxy acids, beta-hydroxy acids, poly-hydroxy acids, benzyl peroxide, zinc salts, sunscreens or mixtures thereof.

Beta-hydroxy acids include salicylic acid, for example. Zinc pyrithione is an example of the zinc salts useful in the skin benefit composition of the present invention. Peroxides, like benzoyl peroxide, may also be employed for effective treatment of acne.

Sunscreens include those materials commonly employed to block ultraviolet light. Illustrative compounds are the derivatives of PABA, cinnamate and salicylate. For example, avobenzophenone (Parsol 1789®) octyl methoxycinnamate and 2-hydroxy-4-methoxyl benzophenone (also known as oxybenzone) can be used. Octyl methoxycinnamate and 2-hydroxy-4-methoxy benzophenone are commercially available under the trademarks, Parsol MCX and Benzophenone-e, respectively. The exact amount of sunscreen employed in the compositions can vary depending upon the degree of protection desired from the sun's UV radiation.

Many skin care compositions, especially those containing water, should be protected against the growth of potentially harmful microorganisms. Anti-microbial compounds, such as triclosan, monoglycerides and/or glycerol monolaurate, and preservatives are, therefore, typically necessary. Suitable preservatives include alkyl esters of p-hydroxybenzoic acid, hydantoin derivatives, propionate salts, and a variety of quaternary ammonium compounds. Particularly preferred preservatives of this invention are methyl paraben, propyl paraben, phenoxyethanol and benzyl alcohol. Preservatives will usually be employed in amounts ranging from about 0.1% to 2% by weight of the composition.

Still other ingredients that may be used with the skin care composition of this invention include vitamins (like niacinamide), recorcinols and retinoids, including retinoic acid, retinal, retinal and retinyl esters.

Typically, when these other ingredients are employed they make up from about 0.001 to about 25%, and preferably, from about 0.01 to about 15%, and most preferably, from about 0.25 to about 10% by weight of the skin care composition, including all ranges subsumed therein. In an especially preferred embodiment, however, the weight in grams of non-volatile fluids used in the skin care composition of this invention is less than the oil absorption number of the spheroid used.

A variety of herbal extracts may optionally be included in compositions of this invention. The extracts may either be water soluble or water-insoluble carried in a solvent which respectively is hydrophilic or hydrophobic. Water and ethanol are the preferred extract solvents. Illustrative extracts include those from green tea, chamomile, licorice, aloe vera, grape seed, citrus unshui, willow bark, sage, thyme and rosemary.

Also included may be such materials as lipoic acid, retinoxytrimethylsilane (available from Clariant Corp. under the Silcare 1M-75 trademark), dehydroepiandrosterone (DHEA) and combinations thereof. Ceramides (including Ceramide 1, Ceramide 3, Ceramide 3B and Ceramide 6) as well as pseudoceramides may also be useful. Amounts of these materials, if used, may range from about 0.000001 to about 10%, preferably from about 0.0001 to about 1% by weight of the composition.

Colorants, opacifiers and abrasives may also be included in compositions of the present invention if desired. Each of these substances may range from about 0.05 to about 5%, preferably between 0.1 and 3% by weight of the composition.

The compositions of the present invention can also be, optionally, incorporated into an insoluble substrate for application to the skin such as in the form of a treated wipe.

A wide variety of packaging can be employed to store and deliver the skin care compositions of this invention. Packaging is often dependent upon the type of personal care end-use. For instance, leave-on skin lotions and creams, shampoos, conditioners and shower gels generally employ plastic containers with an opening at a dispensing end covered by a closure. Typical closures are screw-caps, non-aerosol pumps and flip-top hinged lids. Packaging for antiperspirants, deodorants and depilatories may involve a container with a roll-on ball on a dispensing end. Alternatively these types of skin care compositions may be delivered in a stick composition formulation in a container with propel-repel mechanism where the stick moves on a platform towards a dispensing orifice. Metallic cans pressurized by a propellant and having a spray nozzle serve as packaging for antiperspirants, shave creams and other personal care products. Toilette bars may have packaging constituted by a cellulosic or plastic wrapper or within a cardboard box or even encompassed by a shrink wrap plastic film. All of the aforementioned are considered packaging within context of the present invention. Typically, from about 0.4 to about 1 gram of skin care composition is applied to about 160 cm² of skin.

The Examples are provided to facilitate an understanding of the present invention and they are not meant to limit the scope of the claims.

### EXAMPLE 1

Artificial sebum was made by mixing the following ingredients.

| Ingredient | Weight % |
|---|---|
| Lauric acid | 11.5 |
| Oleic acid | 11.5 |
| Isostearic acid | 5.75 |
| Tricaprin | 11.5 |
| Triolein | 11.5 |
| Glycerol trisostearate | 5.75 |
| Oleyl Oleate | 10.4 |
| Myristyl Myristate | 10.4 |
| Isostearyl Isostearate | 5.2 |
| Squalene | 12 |
| Cholesterol Oleate | 3 |
| Cholesterol | 1.5 |

### EXAMPLE 2

Artificial sebum was combined with thickener and/or spheroid by mixing the same at room temperature as described below.

| | | | | Shear elastic | | | | |
|---|---|---|---|---|---|---|---|---|
| | Thickener | Spheroid | Sebum | modulus Gₒ | | % | | |
| Sample | (wt. %) | (wt. %) | (wt. %) | (Pascals) | P | Wicking | ∈ | Feel |
| 1 | Fumed | DSPCS-I2 | 78.13 | 1.19 × 10³ | 17.7 | 576 | 3.6 | Powdery/silky |
| | silica | (15.63) | | | | | | |
| | 6.25 | | | | | | | |
| 2 | Fumed | DSPCS-I2 | 75.86 | 2.56 × 10⁴ | 17.7 | 576 | 4 | Powdery/silky |
| | silica (6.9) | (17.24) | | | | | | |
| 3 | Fumed | DSPCS-I2 | 72 | 1.12 × 10⁵ | 17.7 | 576 | 5 | Powdery |
| | silica (8) | (20) | | | | | | |
| 4 | — | SPCAT-I2 | 70 | 7.14 × 10⁴ | 16.5 | 1196 | 5.6 | Powdery/silky |
| | | (30) | | | | | | |
| 5 | — | SPCAT-I2 | 67.48 | 5.56 × 10⁵ | 16.5 | 1196 | 6.3 | Powdery/silky |
| | | (32.52) | | | | | | |
| 6 | — | SPCAT-I2 | 65.00 | 8.91 × 10⁵ | 16.5 | 1196 | 7 | Powdery/silky |
| | | (35) | | | | | | |
| 7 | Fumed | CL2080 | 41.2 | 5.7 × 10⁴ | 12 | 2400 | 4.3 | Powdery/silky |
| | silica (2.8) | (56) | | | | | | |
| 8 | — | CL2080 | 40 | 2 × 10³ | 24.5 | 1355 | 4.9 | Silky |
| | | (60) | | | | | | |
| 9* | Fumed | SPCAT-I2 | 76 | 5.54 × 10⁴ | 9 | 924 | 2 | Greasy |
| | silica (12) | (12) | | | | | | |
| 10* | Fumed | SPCAT-I2 | 78 | 4.55 × 10³ | 9 | 924 | 1.8 | Greasy |
| | silica (11) | (11) | | | | | | |
| 11* | Fumed | CL2080 | 86 | 6.5 × 10² | 5.4 | 800 | 0.3 | Oily |
| | silica (7) | (7) | | | | | | |
| 12* | Fumed | CL2080 | 60.38 | 1.4 × 10³ | 12 | 2400 | 2 | Oily |
| | silica (1.9) | (37.7) | | | | | | |
| 13* | — | CL2080 | 50 | 4.2 × 10² | 24.5 | 1355 | 3.2 | Oily |
| | | (50) | | | | | | |
| 14* | — | CL2080 | 50 | 4.2 × 10² | 24.5 | 1355 | 3.2 | Oily |
| | | (50) | | | | | | |
| 15* | Fumed | DSPCS-I2 | 79 | 5 × 10³ | 5 | 224 | 1.6 | Greasy |
| | silica (14) | (7) | | | | | | |

The data in the Table demonstrates that when a skin care composition is made where Gₒ is from about 1×10³ to less than about 9×10⁷ Pascals, and ε is >2.5 but <10, a superior composition that possesses excellent wicking and sensory properties is obtained. The star “*” is meant to identify examples which are comparative.

### EXAMPLE 3

Panelists were asked to wash their faces. Three (3) hours after washing, the panelists were subjected to a sebumeter whereby an oil content value was obtained from their forehead. The panelists having an oil content value of 100 or higher applied compositions with spheroid (10% CL2080 selected in a manner consistent with the criteria described in Sample 8) to their face (about 0.5 to 0.6 grams). Oiliness of the faces was evaluated under two conditions, the first being normal conditions (ambient temperature, about 50% relative humidity) and the second being hot and humid conditions (30° C., about 80% relative humidity). Oiliness perception (look and feel) was scored on a scale of 1 to 9, with 9 being very oily. After three hours of application, panelists with composition made according to this invention had an oiliness rating of 1.5 under normal conditions and 2.3 under hot and humid conditions.

Panelists with a control composition (i.e., compositions not meeting the criteria required in this invention) had an oiliness rating of 3.5 under normal conditions and 4.5 under hot and humid conditions.

What is claimed is:

1. A skin care composition comprising:
(a) 1 to 40% by weight spheroid, the spheroid comprising an approximate diameter from about 3.5 to about 60 microns;
(b) non-volatile fluid;
(c) cosmetically acceptable carrier; and
(d) optionally, a thickening agent,
the spheroid and optionally the thickener when mixed with sebum results in a sebum mixture wherein the spheroid, sebum and optional thickening agent in the mixture each, independently, have a volume fraction at a ratio, E, that is greater than or equal to about 2.5 and less than about 10, where
ε=(C_{B}·V_{B})/(C_{A}/P_{A}+C_{C}/P_{C}),
C_{A} is weight percent of the thickening agent, C_{B} is weight percent of the spheroid, C_{C} is weight percent of sebum plus non-volatile fluid, V_{B} is the inverse of the bulk density of the spheroid (cm³/g), P_{A} is density of the thickening agent, and P_{C} is density of sebum,
and further wherein the mixture has a shear elastic modulus, Gₒ, ranging from about 1×10³ to less than about 9×10⁷ Pascals where the non-volatile fluid is present at a weight percent in grams that is ±15% of an oil absorption number of the spheroid.

2. The skin care composition according to claim 1 wherein the thickener is present.

3. The skin care composition according to claim 1 wherein the spheroid comprises polyethylene, polypropylene, polybutylene, polyamide, or a mixture thereof.

4. The skin care composition according to claim 1 wherein the spheroid comprises polyurethane, polystyrene, epoxy resins, urea resins, silicone resins or a mixture thereof.

5. The skin care composition according to claim 1 wherein the spheroid comprises silica, polystyrene or polylmethylmethacrylate.

6. The skin care composition according to claim 1 wherein the spheroid has an oil absorption number of 0.2 to 15 g/g.

7. The skin care composition according to claim 1 wherein the spheroids make up from about 3 to about 15 percent by weight of the skin care composition.

8. The skin care composition according to claim 1 wherein Gₒ is from about 4×10³ to about 5×10⁵ pascals.

9. The skin care composition according to claim 1 wherein the spheroid has an oil absorption number from about 0.2 to about 12 g/g.

10. The skin care composition according to claim 1 wherein the spheroids comprise an approximate diameter from about 4 to about 40 microns.

11. The skin care composition according to claim 1 wherein the skin care composition is not greasy and is powdery and silky when applied.

12. The skin care composition according to claim 1 wherein a 37 micron film of the skin care composition is suitable to wick 1 microliter of sebum at an initial volume Aⁱ to a final volume A^{f}, where A^{f} is at least about 250% greater than Aⁱ after about 30 minutes.

13. A method for regulating sebum flow on skin comprising the step of applying to the skin care composition comprising
(a) 1 to 40% by weight spheroid, the spheroid comprising an approximate diameter from about 3.5 to about 60 microns;
(b) non-volatile fluid;
(c) cosmetically acceptable carrier; and
(d) optionally, a thickening agent,
the spheroid and optionally the thickener when mixed with sebum results in a sebum mixture wherein the spheroid, sebum and optional thickening agent in the mixture each, independently, have a volume fraction at a ratio, ε, that is greater than or equal to about 2.5 and less than about 10, where
ε=(C_{B}·V_{B})/C_{A}/P_{A}+C_{C}/P_{C}),
C_{A} is weight percent of the thickening agent, C_{B} is weight percent of the spheroid, C_{C} is weight percent of sebum plus non-volatile fluid, V_{B} is the inverse of the bulk density of the spheroid (cm³/g), P_{A} is density of the thickening agent, and P_{C} is density of sebum,
and further wherein the mixture has a shear elastic modulus, Gₒ, ranging from about 1×10³ to less than about 9×10⁷ Pascals where the non-volatile fluid is present at a weight percent in grams that is ±15% of an oil absorption number of the spheroid.

14. The method for regulating sebum flow on skin according to claim 13 wherein Gₒ is from about 4×10³ to about 5×10⁵ pascals.

15. The method for regulating sebum flow on skin according to claim 14 wherein the spheroid has an oil absorption number from about 0.2 to about 12 g/g.
